(19) United States
(12) Patent Application Publication
(10) Pub. No.: US 2019/0004308 A1
(43) Pub. Date: Jan. 3, 2019

(54) Ultra superfine imaging unit and videoscope

(71) Applicant: MPI Inc., Tokyo (JP)

(72) Inventor: Takeshi Iwama, Tokyo (JP)

(21) Appl. No.: 15/999,213

(22) Filed: Aug. 16, 2018

Related U.S. Application Data
(63) Continuation of application No. PCT/JP2016/074235, filed on Aug. 16, 2016.

(30) Foreign Application Priority Data
Feb. 16, 2016 (JP) 2016-026899

(51) Int. Cl. G02B 23/24; A61B 1/07; A61B 1/05; G02B 23/26; A61B 1/00

(52) U.S. Cl. CPC G02B 23/2469; A61B 1/07; G02B 23/2484; G02B 23/26; A61B 1/00183; A61B 1/05

(57) ABSTRACT

An ultra superfine imaging unit 26 is applicable to an insertion tip portion 24a of a videoscope 1. The imaging unit includes a case main body 27 having a shape of a hollow cylinder and arrangeable in the insertion tip portion, at least one light emitting portion 28 accommodated in the case main body and configured to output light to an observation object, and a light receiving portion 29 accommodated in the case main body and configured to receive input of an image from the observation object illuminated with light. Inside the case main body, the light emitting portion is arranged along a circumference of the light receiving portion.

### CROSS-REFERENCE TO RELATED APPLICATIONS

This application is a Continuation Application of PCT Application No. PCT/JP2016/074235, filed Aug. 19, 2016 and based upon and claiming the benefit of priority from prior Japanese Patent Application No. 2016-026899, filed Feb. 16, 2016, the entire contents of all of which are incorporated herein by reference.

### BACKGROUND OF THE INVENTION

### 1. Field of the Invention

The present invention relates to an ultra superfine imaging unit applicable to an insertion tip portion of every videoscope used not only in the medical field but also in the industrial field, in particular, an ultra superfine imaging unit where an external size of an insertion tip portion can be set to an ultra super small size of several millimeter or less (for example, a diameter of 2 mm).

In the specification and the claims of the present application, the “insertion tip portion” indicates an end region (that is, a tip portion and its neighboring region) of the videoscope. For example, the end region (insertion tip portion) is a portion which can be inserted (advanced) toward an observation object, and can be arranged closely and oppositely to the observation object.

### 2. Description of the Related Art

In the medical field and the industrial field, conventionally, a fiberscope has been mainly used as a tool for observing an observation object (for example, the inside of a hole). On the other hand, recently, a high resolution videoscope has been mainly used in place of the fiberscope as disclosed, for example, in Patent Literature 1 (the patent applicant: Olympus Corporation).

In a videoscope of Patent Literature 1, an imaging unit is provided in an insertion tip portion. A light source device, a video processor and a monitor are connected to the imaging unit. In this structure, illumination light is supplied from the light source device to the imaging unit. Then, illumination light is emitted from the imaging unit toward an observation object. At this time, the imaging unit converts an image of the observation object into an electric signal. The video processor applies image processing to the electric signal. In this way, the image of the observation object is displayed in color on the monitor based on the output of the video processor.

### CITATION LIST

### Patent Literature

Patent Literature 1: JP 2007-289278 A

### BRIEF SUMMARY OF THE INVENTION

In the meantime, in the above-described videoscope, improvement of the resolution and reduction of the external size (for example, the diameter) as much as possible are required of the imaging unit of the insertion tip portion. However, in the conventional technology, the resolution improvement and the size reduction of the imaging unit could not have been achieved at the same time. This is because of the following reason.

That is, the imaging unit includes, for example, a light emitting portion configured to output light to an observation object, and a light receiving portion configured to receive input of an image of an observation object illuminated with light. In the light emitting portion, for example, an optical fiber, an LED and the like are arranged. In the light receiving portion, for example, an objective lens, a sensor (a CCD sensor or a CMOS sensor) and the like are arranged.

In this structure, to reduce the size of the imaging unit, for example, the sizes of the light emitting portion and the light receiving portion need to be reduced. In that case, as the sizes are reduced, the optical performance (for example, illumination intensity (brightness)) of the light emitting portion and the optical performance (for example, light sensitivity) of the light receiving portion will be degraded, accordingly. As a result, the resolution of the imaging unit will be reduced.

On the other hand, to improve the resolution of the imaging unit, for example, a broad light emitting region of the light emitting portion and a broad light receiving region of the light receiving portion need to be secured. In that case, as these regions are expanded, the light emitting portion and the light receiving portion will be increased, accordingly. As a result, the size of the imaging unit will be increased.

As described above, the resolution improvement and the size reduction of the imaging unit have an antinomy relation. In this case, the antinomy relation cannot be solved by simply combining the light emitting portion and the light receiving portion. In other words, the relation can be solved by elaborating the layout of the light emitting portion and the light receiving portion. However, an imaging unit having an elaborated layout of a light emitting portion and a light receiving portion is not known at this moment.

An object of the present invention is to provide an ultra superfine imaging unit which can achieve both the resolution improvement and the size reduction of the imaging unit by elaborating the layout of a light emitting portion and a light receiving portion.

To attain this object, the present invention is directed to an ultra superfine imaging unit which is applicable to an insertion tip portion of a videoscope and in which the insertion tip portion has an external size settable to an ultra small size of several millimeters or less, and the ultra superfine imaging unit includes a case main body having a shape of a hollow cylinder and arrangeable in the insertion tip portion, at least one light emitting portion accommodated in the case main body and configured to output light to an observation object, and a light receiving portion accommodated in the case main body and configured to receive input of an image from the observation object illuminated with light, and inside the case main body, the light emitting portion is arranged along a circumference of the light receiving portion.

According to the present invention, an ultra superfine imaging unit which can achieve both the resolution improvement and the size reduction of the imaging unit by elaborating the layout of a light emitting portion and a light receiving portion can be achieved.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view of a videoscope adopting an ultra superfine imaging unit according to one embodiment of the present invention.

FIG. 2 is a sectional view of the ultra superfine imaging unit taken along line F2-F2 of FIG. 1.

FIG. 3 is a side view of a state where a video unit of FIG. 1 is rotated.

FIG. 4 is an end view of the structure of an ultra superfine imaging unit according to one modification of the present invention.

FIG. 5 is an end view of the structure of an ultra superfine image unit according to another modification of the present invention.

### DETAILED DESCRIPTION OF THE INVENTION

### One Embodiment

“Regarding Videoscope”

An ultra superfine imaging unit (hereinafter referred to as an imaging unit) of the present embodiment is applicable to an insertion tip portion of a videoscope. As an example of the videoscope, various videoscopes used in the industrial field and the medical field can be assumed. As the videoscope of the industrial field, for example, an industrial endoscope which enables observation of an observation object in a place at a construction site which is too narrow for a construction worker to enter can be assumed. On the other hand, as the videoscope of the medical field, for example, a medical endoscope which enables observation of an observation object in a patient's body can be assumed.

The “insertion tip portion” here indicates an end region (that is, an end portion and its neighboring region) of the above-described videoscope. For example, the end region (insertion tip portion) is a portion which can be inserted (advanced) toward an observation object, and can be arranged closely and oppositely to the observation object.

Here, as the videoscope of the medical field, for example, a flexible videoscope having a flexible insertion portion, a rigid videostylet having a rigid insertion portion, a laryngoscope blade, an otoscope, an anoscope and the like can be assumed. As an example of the videoscope of the medical field, a flexible videoscope is shown in the drawing. The flexible videoscope will be described below.

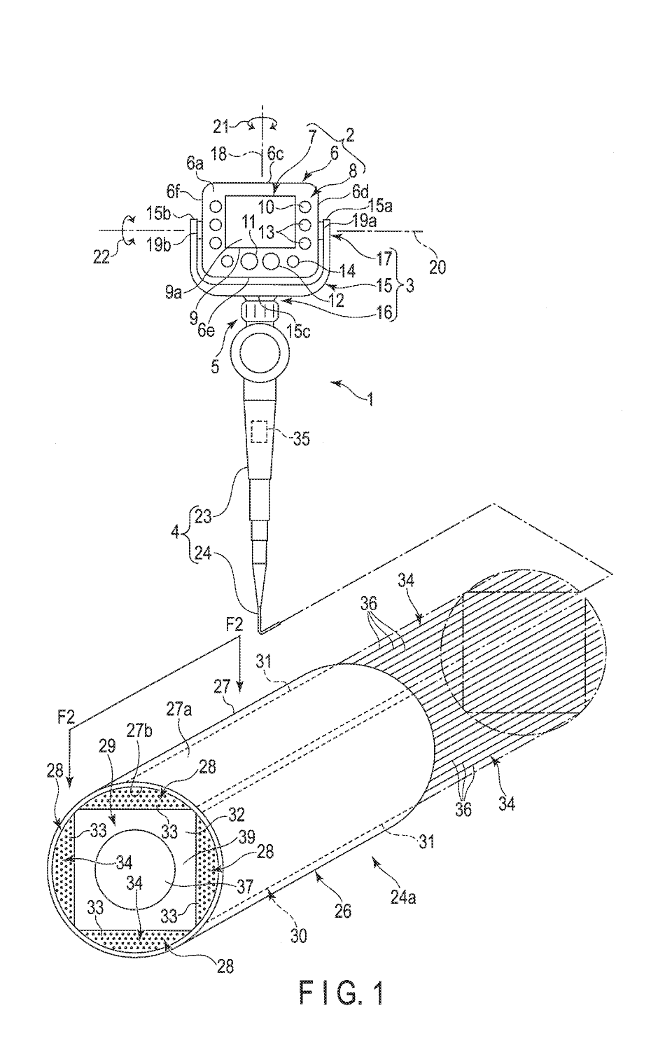

As shown in FIG. 1, a flexible videoscope 1 includes a video unit 2, an angle adjustment unit 3, a scope unit 4 and a connection mechanism 5. The video unit 2 is rotatably supported on the angle adjustment unit 3. The angle adjustment unit 3 is rotatably connected to the scope unit 4 by the connection mechanism 5. In this way, the video unit 2 is configured to rotate in desired directions (for example, a rolling direction 21 and a yawing direction 22 which will be described later). Specific descriptions thereof will be presented below.

“Video Unit 2”

As shown in FIGS. 1 and 3, the video unit 2 includes a video main body portion 6, a display portion 7 and an operation portion 8.

The display portion 7 and the operation portion 8 are provided in the video main body portion 6. The whole video unit 2 (that is, the video main body portion 6) has a waterproof structure or a dustproof structure.

The video main body portion 6 has two wide surfaces (a front surface 6a and a back surface 6b) and four narrow surfaces (a first surface 6c, a second surface 6d, a third surface 6e and a fourth surface 6f). The front surface 6a and the back surface 6b are opposed to each other. The front surface 6a and the back surface 6b have substantially the same size and shape as each other. Here, the front surface 6a and the back surface 6b which are formed in the shape of a wide rectangle are assumed as an example.

The first to fourth surfaces 6c to 6f are arranged between the front surface 6a and the back surface 6b. Between the front surface 6a and the back surface 6b, the first to fourth surfaces 6c to 6f are continuously arranged in this order. In this arrangement, the first surface 6c and the third surface 6e are opposed to each other in parallel. The second surface 6d and the fourth surface 6f are opposed to each other in parallel. Further, the second surface 6d and the fourth surface 6f are continuous on both sides of the first surface 6c. The second surface 6d and the fourth surface 6f are continuous on both sides of the third surface 6e. In other words, the first surface 6c and the third surface 6e are continuous on both sides of the second surface 6d. The first surface 6c and the third surface 6e are continuous on both sides of the fourth surface 6f.

The first surface 6c and the third surface 6e have substantially the same size and shape as each other. The second surface 6d and the fourth surface 6f have substantially the same size and shape as each other. Here, the first to fourth surfaces 6c to 6f which are formed in the shape of a narrow rectangle are assumed as an example.

A monitor 9 is mounted on the video main body portion 6. The monitor 9 is a display device configured to display a color image (a moving image or a still image). As the display device, for example, a liquid crystal display (LCD), an organic EL display or the like can be applied.

Further, although an illustration is not given in particular in the drawing, a power supply unit, a control unit and the like are accommodated in the video main body portion 6, for example. The power supply unit is equipped with a replaceable battery. The power supply unit can supply electric power to the monitor 9, a light source 35 (for example, an LED) of an imaging unit 26 which will be described later and the like, for example. The control unit can control the monitor 9 and the power supply unit, and the operation portion 8 which will be described later, for example.

Furthermore, in the video main body portion 6, the display portion 7 and the operation portion 8 are arranged on the front surface 6a.

The display portion 7 is arranged at the center of the front surface 6a. The above-described monitor 9 is arranged in the display portion 7. The monitor 9 has a wide display screen 9a along the front surface 6a of the video main body portion 6. The display screen 9a is exposed to the outside through the display portion 7.

The operation portion 8 is arranged along the circumference of the display portion 7. The operation portion 8 has a plurality of buttons. As the buttons, for example, a power button 10, a moving image save button 11, a still image save button 12, a light source control button 13, an image reproduction button 14 and the like can be assumed.

In the above-described structure, the power button 10 is set to an on state. By this operation, the color image (the moving image or the still image) of the observation object captured by the imaging unit 26 which will be described later can be displayed on the monitor 9 (the display screen 9a). As a result, while operating the flexible videoscope 1, the user can visually check the observation object displayed in color on the monitor 9 (the display screen 9a) via the display portion 7 in real time.

At this time, the moving image save button 11 or the still image save button 12 is set to an on state, for example. By this operation, the color image (the moving image or the still image) of the observation object displayed on the monitor 9 (the display screen 9a) at the present moment can be saved in real time. As the save destination, although an illustration is not given in particular in the drawing, an internal memory (for example, a RAM) of the video main body portion 6 or an external memory (for example, an SD card) can be applied, for example.

Here, the image reproduction button 14 is set to an on state, for example. By this operation, the already-saved color image (the moving image or the still image) can be displayed on the monitor 9 (the display screen 9a). As a result, the user can check all parts of the observation object again or only desired parts of the observation object again, for example.

“Angle Adjustment Unit 3”

The angle adjustment unit 3 includes a support frame 15, a rolling mechanism 16 and a yawing mechanism 17.

The support frame 15 is shaped in such a manner as to surround the outside of the video main body portion 6. The support frame 15 has both ends (one end 15a and the other end 15b) and a central portion 15c. In the support frame 15, the central portion 15c is rotatably connected to the scope unit 4 which will be described later by the connection mechanism 5.

The rolling mechanism 16 includes one support member (not shown). The support member is provided between the central portion 15c of the support frame 15 and the connection mechanism 5. The support member is rotatable around a single first rotation axis 18 in the rolling (right-and-left) direction 21. In this way, the support frame 15 is rotatably supported on the scope unit 4 which will be later by the support member (refer to FIG. 1).

The yawing mechanism 17 includes two support pins 19a and 19b. These two support pins 19a and 19b are provided at the one end 15a and the other end 15b of the support frame 15, respectively. These support pins 19a and 19b are arranged in positions which are opposed to each other in parallel. These support pins 19a and 19b are rotatable around a single second rotation axis 20 in the yawing (up-and-down) direction 22. The second rotation axis 20 has such a positional relationship that the second rotation axis 20 is orthogonal to the above-described first rotation axis 18.

The one end 15a of the support frame 15 is parallel to the second surface 6d of the video main body portion 6. The one support pin 19a is provided between the one end 15a of the support frame 15 and the second surface 6d of the video main body portion 6. In this way, the second surface 6d of the video main body portion 6 is rotatably supported on the one end 15a of the support frame 15 by the one support pin 19a.

The other end 15b of the support frame 15 is parallel to the fourth surface 6f of the video main body portion 6. The other support pin 19b is provided between the other end 15b of the support frame 15 and the fourth surface 6f of the video main body portion 6. In this way, the fourth surface 6f of the video main body portion 6 is rotatably supported on the other end 15b of the support frame 15 by the other support pin 19b. Accordingly, the video main body portion 6 is rotatably supported on the support frame 15 by the above-described two support pins 19a and 19b (refer to FIG. 3).

According to the angle adjustment unit 3, the video main body portion 6 is configured to rotate with respect to the scope unit 4 which will be described later not only in the rolling (right-and-left) direction 21 but also in the yawing (up-and-down) direction 22. Therefore, the video main body portion 6 can be rotated or swung freely in a range of 360 degrees. As a result, the orientation of the monitor 9 (the display screen 9a) of the video main body portion 6 can be freely adjusted to a user's easily viewable angle (for example, an angle shown in FIG. 3).

“Scope Unit 4”

The scope unit 4 includes a scope main body portion 23 and an insertion portion 24 which can be inserted into the body.

Although an illustration is not given in particular in the drawing, an operation lever, an operation button and the like are provided in the scope main body portion 23. Furthermore, the light source 35 (for example, an LED) of the imaging unit 26 which will be described later is accommodated in the scope main body portion 23. Note that the accommodation place of the light source 35 (for example, an LED) is not limited to the scope main body portion 23 but may be set to another place (for example, the above-described video main body portion 6).

The insertion portion 24 has flexibility. The imaging unit 26 will be described later is provided in the end region of the insertion portion 24, that is, the tip portion and the neighboring region (hereinafter referred to as an insertion tip portion 24a). In this structure, for example, the insertion tip portion 24a can be bent toward the observation object by operating the operation lever.

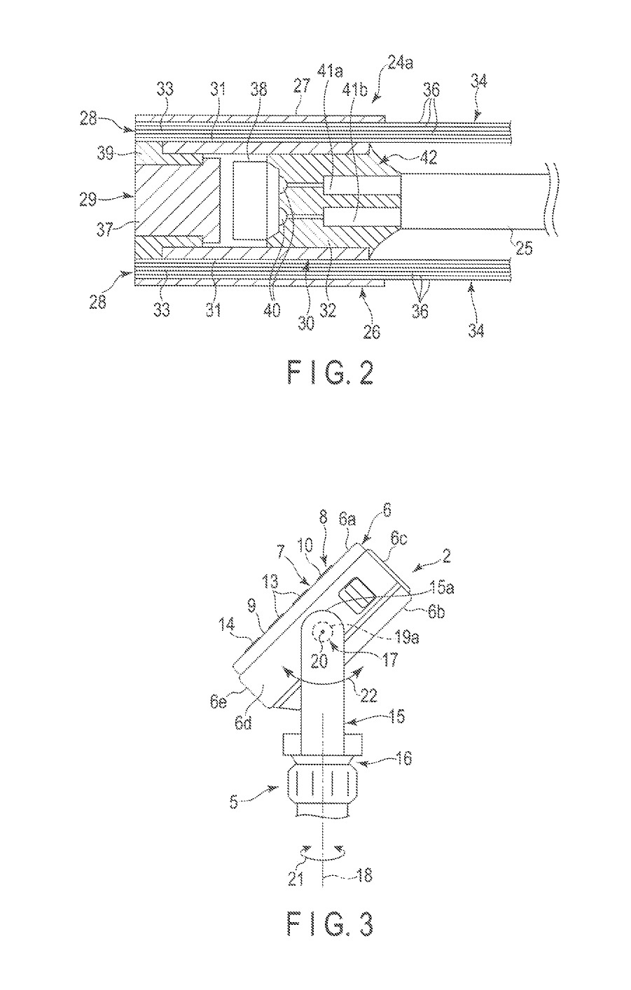

Inside the insertion portion 24, a cable 25 (for example, a power supply line 41a) for supplying electric power to the imaging unit 26 which will be described later, a cable 25 (for example, a signal line 41b) for transmitting an output signal of the imaging unit 26 to the video unit 2 (the video main body portion 6) are provided (refer to FIG. 2). The electric power is supplied from the power supply unit (not shown) of the above-described video unit 2 (the video main body portion 6).

“Connection Mechanism 5”

The connection mechanism 5 is configured to detachably connect the angle adjustment unit 3 and the scope unit 4. In this way, the one video unit 2 can be applied not only to the above-described flexible videoscope 1 but also to various other videoscopes (for example, a rigid video stylet, a laryngoscope blade, an otoscope, an anoscope, etc.), selectively. As the connection mechanism 5, for example, a commercially-available well-known fastener (not shown) can be directly used. Therefore, description of the specification of the connection mechanism will be omitted. “Regarding Imaging Unit 26”

As shown in FIGS. 1 and 2, the imaging unit 26 is applied to the insertion tip portion 24a of the flexible videoscope 1 (the insertion portion 24). In the imaging unit 26, the external size of the insertion tip portion 24a can be set to an ultra super small size of several millimeters or less. Here, as the shape of a cross-section of the insertion tip portion 24a, various shapes such as an elliptical shape, a circular shape, a triangular shape and a quadrangular shape can be assumed. In the drawing, the imaging unit 26 having a circular cross-section is shown as an example.

As shown in FIGS. 1 and 2, the imaging unit 26 includes one case main body 27, a plurality of light emitting portions 28, one light receiving portion 29 and one holder 30.

The case main body 27 has the shape of a hollow cylinder which can be arranged in the insertion tip portion 24a. An outer circumferential surface 27a of the case main body 27 has the shape of a cylinder which is smooth and has no irregularities. Therefore, the insertion tip portion 24a can be smoothly inserted into the body, and the insertion tip portion 24a can also be smoothly pulled out from the body.

An inner circumferential surface 27b of the case main body 27 has the shape of a cylinder having no irregularities. Therefore, the internal space (accommodation space) of the case main body 27 can be maximized. As a result, all the structures of the light emitting portions 28, the light receiving portion 29 and the holder 30 can be accommodated in the internal space (accommodation space) of the single case main body 27.

Here, to realize both the resolution improvement and the size reduction of the imaging unit 26, in the internal space (accommodation space) of the case main body 27, the layout of the light emitting portion 28 and the light receiving portion 29 needs to be elaborated. In this case, the plurality of light emitting portions 28 should preferably be arranged along the circumference of the light receiving portion 29 at regular intervals and concentrically in the internal space (accommodation space) of the case main body 27.

“Optimum Layout”

The holder 30 is configured to hold the light emitting portions 28 and the light receiving portion 29 in the internal space (accommodation space) of the case main body 27. Therefore, the holder 30 is configured to divide the internal space (accommodation space) of the case main body 27 into divisions. In the drawing, the holder 30 has the shape of a hollow square prism as an example. The holder 30 has a square cross-sectional contour.

In this case, the holder 30 includes four wall portions 31. The four wall portions 31 are composed of two pairs of wall portions 31, each pair of which are opposed to each other in parallel. The four wall portions 31 have such a positional relationship that the adjacent wall portions 31 are orthogonal to each other. All the wall portions 31 have the same size and shape as each other. Here, the rectangular (for example, thin plate-like rectangular) wall portions 31 are assumed as an example.

In this structure, in a state where the holder 30 is accommodated in the case main body 27, the inside of the case main body 27 is divided into one first region 32 and four second regions 33 arranged along the circumference of the first region 32 at regular intervals.

The first region 32 has a square cross-sectional contour surrounded by the four wall portions 31. The light receiving portion 29 is accommodated in the first region 32. The four second regions 33 have a circular arc cross-sectional contour surrounded respectively by the wall portions 31 and the case main body 27 (the inner circumferential surface 27b). The four light emitting portions 28 are accommodated in the four second regions 33, respectively.

According to this layout, light required and sufficient for illuminating an observation object can be output from the four light emitting portions 28. Therefore, the image of an observation object illuminated with light can be accurately input to the light receiving portion 29. As a result, the resolution of the imaging unit 26 can be improved.

According to this layout, the four light emitting portions 28 and the light receiving portion 29 can be closely arranged to each other. Therefore, the cross-sectional area occupied with all the structures from the light receiving portion 29 to the four light emitting portions 28 can be reduced as much as possible. In this case, as the cross-sectional area is reduced, the external size (for example, the diameter) of the case main body 27 can be reduced, accordingly. As a result, the size of the imaging unit 26 can be reduced.

“Light Emitting Portion 28 and Light Receiving Portion 29”

The light emitting portion 28 is configured to output light to an observation object. A light guide 34 which has both ends and the light source 35 are connected to the light emitting portion 28. As the light source 35, for example, an LED can be applied. The light guide 34 can be composed of one or a plurality of optical fibers 36. In the drawing, the light guide 34 is composed of a plurality of optical fibers 36 as an example.

The one end of the light guide 34 (the optical fibers 36) is accommodated in the case main body 27. The other end of the light guide 34 (the optical fibers 36) is configured such that light from the light source 35 can be input. In this structure, the light input to the other end is transmitted along the light guide 34 (the optical fibers 36) while repeating total internal reflection and is eventually output from the one end.

The light receiving portion 29 is configured such that an image from an observation object illuminated with light can be input. The light receiving portion 29 includes an objective lens 37 and a sensor 38.

The objective lens 37 is fixed to the holder 30 by a fixture 39.

As the sensor 38, for example, a CMOS image sensor, a CCD image sensor or the like can be applied. The sensor 38 is fixed to the holder 30 by a fixture (not shown). The above-described power supply line 41a and signal line 41b are directly connected to the sensor 38 by solder 40.

The inside of the case main body 27 is tightly filled with a mold material 42. The mold material 42 covers the whole solder 40. The mold material 42 covers the whole power supply line 41a and the whole signal line 41b which are connected to the sensor 38 by the solder 40. The mold material 42 is formed of a material excellent in durability, water resistance and heat resistance.

In this structure, electric power is supplied from the above-described power supply unit (not shown) to the sensor 38 via the cable 25 (that consists of the power supply line 41a and signal line 41b). Here, the image of an observation object is input to the sensor 38 (an image sensor) via the objective lens 37. Subsequently, information about the image (for example, a shape, a size, a color, etc.) is converted into an electric signal by the sensor 38 (an image sensor).

At this time, the electric signal output from the sensor 38 (an image sensor) is transmitted to the video unit 2 (the video main body portion 6) via the above-described cable 25 (that consists of the power supply line 41a and signal line 41b). In this way, the color image (the moving image or the still image) of an observation object is displayed on the monitor 9 (the display screen 9a).

“Effect of One Embodiment”

According to the present embodiment, the holder 30 having the shape of a hollow square prism (square cross-sectional contour) is arranged in the internal space (accommodation space) of the case main body 27. Consequently, in the internal space (accommodation space) of the case main body 27, the four second regions 33 are arranged along the circumference of the one first region 32 at regular intervals and concentrically. Further, the light receiving portion 29 is accommodated in the first region 32. The four light emitting portions 28 are accommodated in the four second regions 33, respectively. According to this layout, both the resolution improvement and the size reduction of the imaging unit 26 can be achieved.

That is, according to this layout, light required and sufficient for illuminating the observation object can be simultaneously output from the four light emitting portions 28. In this case, a light receiving region of a required and sufficient size can be secured in the light receiving portion 29. Therefore, the image of an observation object illuminated with light can be accurately input to the light receiving portion 29. As a result, the resolution of the imaging unit 26 can be improved.

Further, according to this layout, the four light emitting portions 28 and the light receiving portion 29 can be most efficiently, closely arranged to each other. Therefore, the cross-sectional area occupied with all the structures from the light receiving portion 29 to the four light emitting portions 28 can be reduced as much as possible. In this case, as the cross-sectional area is reduced, the external size of the case main body 27 can be reduced, accordingly. As a result, the size of the imaging unit 26 can be reduced.

According to the present embodiment, in the above-described layout, the first region 32 accommodating the light receiving portion 29 is formed to have a square cross-sectional contour, and the second regions 33 accommodating the light receiving portions 28 is formed to have a circular arc cross-sectional contour. Therefore, both the resolution improvement and the size reduction of the imaging unit 26 can be achieved.

According to the present embodiment, the inside of the case main body 27 is tightly filled with the mold material 42 excellent in durability, water resistance and heat resistance. Therefore, the whole solder 40 can be covered with the mold material 42. Further, the whole power supply line 41a and the whole signal line 41b which are connected to the sensor 38 by the solder 40 can be covered with the mold material 42.

According to this structure, for example, if the insertion tip portion 24a is bent toward an observation object, stress concentration on the connection portions between the sensor 38 and the power supply line 41a and the signal line 41b can be prevented. That is, external forces will not be intensively applied to the connection portions. Therefore, occurrence of trouble such as detachment of the power supply line 41a and the signal line 41b from the sensor 38 can be prevented beforehand. In this case, the connection of the connection portions can be maintained for a long period of time. As a result, the life of the imaging unit 26 can be extended.

Furthermore, according to this structure, all the electric structures inside the case main body 27 can be covered airtightly or fluidtightly with the mold material 42. Therefore, for example, even in the case of observing the inside of the body or even in the case of sterilizing the whole insertion portion 24 including the insertion tip portion 24a, moisture of body fluid, an antiseptic solution or the like can be prevented from entering inside the case main body 27 beforehand. As a result, occurrence of trouble such as a short circuit and an electric shock can be prevented beforehand, and for example, early deterioration of the electric structures of the sensor 38, the power supply line 41a, the signal line 41b and the like caused by oxidation can also be prevented beforehand.

In the present embodiment, according to the above-described layout, in a state where the case main body 27 is arranged in the insertion tip portion 24a, the external size (for example, the diameter) of the insertion tip portion 24a can be set to a range of 1.6 mm to 3.2 mm, more preferably, 2 mm. Therefore, the insertion tip portion 24a can be smoothly inserted into the body, and the insertion tip portion 24a can also be smoothly pulled out from the body.

In the above-described embodiment, although reference was not made in particular to the total length of the insertion tip portion 24a, for example, when operativity and insertability in the patient's body are taken into consideration, the total length of the insertion tip portion 24a should preferably be set to a range of 3.0 mm to 4.0 mm. In this case, the total length of the insertion tip portion 24a indicates the length in the insertion direction of the flexible videoscope 1.

“Modification”

The present invention is not limited to the above-described embodiment, and the following modifications are also included in the scope of the technical idea of the present invention. The inventions according to the modifications can produce the same effect as that of the above-described embodiment. Therefore, description of the effect will be omitted.

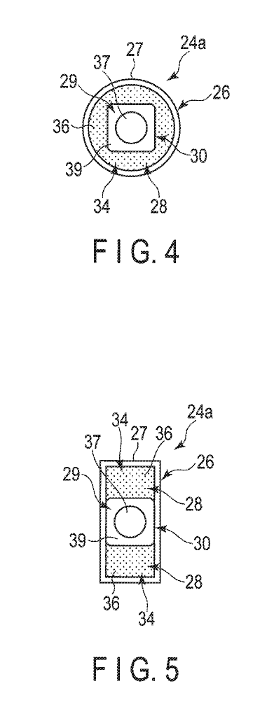

In the modification shown in FIG. 4, one light emitting portion 28 is continuously and concentrically arranged along the circumference of the light receiving portion 29. In this case, the external size (the diameter) of the insertion tip portion 24a is set to 2.5 mm. Note that, since the other structures are the same as those of the above-described embodiment, descriptions thereof are omitted.

In the modification shown in FIG. 5, a plurality of light emitting portions 28 are arranged along the circumference of the light receiving portion 29 at regular intervals. In this modification, two light emitting portions 28 are arranged in such a manner as to be opposed to both sides of the light receiving portion 29, respectively. For this reason, the insertion tip portion 24a has a quadrangular or rectangular cross-section. In this case, the external sizes of the insertion tip portion 24a are set to 3.2 mm in the longitudinal direction and 1.6 mm in the lateral direction. Note that, since the other structures are the same as those of the above-described embodiment, descriptions thereof are omitted.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

### REFERENCE SIGNS LIST

1 . . . flexible videoscope, 2 . . . video unit, 3 . . . angle adjustment unit, 4 . . . scope unit, 5 . . . connection mechanism, 26 . . . imaging unit, 27 . . . case main body, 28 . . . light emitting portion, 29 . . . light receiving portion, 30 . . . holder, 34 . . . light guide, 35 . . . light source, 36 . . . optical fiber, 37 . . . objective lens.

What is claimed is:

1. An ultra superfine imaging unit applicable to an insertion tip portion of a videoscope, the insertion tip portion having an external size settable to an ultra small size of several millimeters or less, the ultra superfine imaging unit comprising:
a case main body having a shape of a hollow cylinder and arrangeable in the insertion tip portion;
at least one light emitting portion accommodated in the case main body and configured to output light to an observation object; and
a light receiving portion accommodated in the case main body and configured to receive input of an image from the observation object illuminated with light, wherein
the light emitting portion is arranged along a circumference of the light receiving portion inside the case main body, further comprising
a holder having a shape of a hollow square prism and configured to hold the light emitting portion and the light receiving portion inside the case main body, wherein
the holder comprises four wall portions opposed to each other in parallel in such a manner as to divide an inside of the case main body, and adjacent wall portions of the four wall portions are orthogonal to each other,
the inside of the case main body is divided into a first region and a second region arranged along a circumference of the first region in a state where the holder is accommodated in the case main body,
the first region has a square cross-sectional contour surrounded by the four wall portions,
the second region has a cross-sectional contour surrounded by the walls and the case main body,
the light receiving portion is accommodated in the first region, and
the light emitting portion is accommodated in the second region.

2. The ultra superfine imaging unit of claim 1, wherein the plurality of light emitting portions are arranged along the circumference of the light receiving portion at regular intervals and concentrically.

3. The ultra superfine imaging unit of claim 1, wherein the one light emitting portion is arranged along the circumference of the light receiving portion continually and concentrically.

4. The ultra superfine imaging unit of claim 1, wherein the plurality of light emitting portions are arranged along the circumference of the light receiving portion at regular intervals.

5. The ultra superfine imaging unit of claim 1, wherein the external size of the insertion tip portion is settable to a range of 1.6 mm to 3.2 mm in a state where the case main body is arranged in the insertion tip portion.

6. The ultra superfine imaging unit of claim 5, wherein a total length of the insertion tip portion in an insertion direction of the videoscope is set to a range of 3.0 mm to 4.0 mm.

7. The ultra superfine imaging unit of claim 1, wherein
a light guide having both ends and a light source are connected to the light emitting portion,
the one end of the light guide is accommodated in the case main body,
the other end of the light guide is configured to receive input of light from the light source,
light input to the other end is transmitted along the light guide and is output from the one end.

8. The ultra superfine imaging unit of claim 7, wherein the light guide is formable of one or a plurality of optical fibers.

9. The ultra superfine imaging unit of claim 1, wherein
an objective lens and a sensor are provided in the light receiving portion, and
the image of the observation object is input to the sensor via the objective lens and is converted into an electric signal by the sensor.

10. The ultra superfine imaging unit of claim 1, wherein
the case main body has an inner circumferential surface and an outer circumferential surface, and
the inner circumferential surface and the outer circumferential surface have a shape of a cylinder having no irregularities.

11. A videoscope comprising the ultra superfine imaging unit of any one of claims 1 to 10, the videoscope comprising:
a video unit configured to display a color image of the observation object on a display screen;
an angle adjustment unit configured to adjust the display screen to a user's easily viewable angle;
a scope unit comprising an insertion portion insertable into a body; and
a connection mechanism configured to detachably connect the angle adjustment unit and the scope unit, wherein
the ultra superfine imaging unit is applied to the insertion tip portion of the insertion portion.
